(19) United States
(12) Patent Application Publication
(10) Pub. No.: US 2002/0187185 A1
(43) Pub. Date: Dec. 12, 2002

(54) Gelatin substitute

(76) Inventor: Roger Trevor Jones, Cheshire (GB)

Correspondence Address: STEVENS, DAVIS, MILLER & MOSHER, LLP, Suite 850, 1615 L. Street, N.W., Washington, DC, 20036, US

(21) Appl. No.: 10/141,346

(22) Filed: May 9, 2002

(30) Foreign Application Priority Data
May 10, 2001 (GB) 0111402.4

(51) Int. Cl. A61K 9/48; A61K 9/16; A61K 9/50

(52) U.S. Cl. 424/452; 424/491

(57) ABSTRACT

The use of a protein of vegetable origin suitable in capsule or microcapsule manufacture, which protein

(a) has a molecular weight of at least 40 kD; and

(b) is water soluble, whereby a clear aqueous solution can be formed that can produce a clear film on drying.

This invention relates to new vegetable protein-derived materials which have good physical properties and may be used to replace gelatin in a diverse range of applications, especially in pharmaceutical capsule manufacture.

Gelatin is a hydrocolloid, being a substance that forms a colloidal solution in water, which exhibits a unique combination of useful properties. These properties include water solubility, solution viscosity, thermally-reversible gelation properties and an ability to form strong, clear, flexible, high-gloss films. Moreover, the gels melt at body temperature and films will dissolve when digested. Gelatin is also a natural product, and as a protein it is classified as a food rather than a food additive.

Commercial uses for gelatin have been established in a wide range of industries, including applications in food, pharmaceutical, medical, photographic, cosmetic and technical products. Commercially, one of the major applications for gelatin is in the pharmaceutical industry, in the production of hard and soft capsules, where the ability of gelatin to form clear, flexible, glossy capsule walls is important. The ability of the gelatin capsules to dissolve in the stomach can also be necessary. Gelatin is also used for the micro-encapsulation of oils and vitamins (especially vitamins A and E) for edible and pharmaceutical uses.

Gelatin is available in various grades and, in turn, has different average molecular weights. Commercially, gelatins tend to be graded in terms of their gel strengths (Bloom value) under standard test conditions, although viscosity is generally also an important parameter for encapsulation applications. For such applications, gelatins will typically have Bloom gel strengths in the range 100-280 g and viscosities (tested on 6.67% solution at 60° C.) in the range 2.0-5.5 mPas. Molecular weight values are not normally cited, since there is no universally accepted test procedure for gelatin and the values for average molecular weights can vary dependent on the test method and procedure used. However, based on a size exclusion HPLC method, the above-mentioned gelatins typically have weight average molecular weights in the range 80,000-200,000 Daltons. Lower molecular weight gelatins are available and non-gelling versions can be produced by deliberately hydrolysing the gelatins down to weight average molecular weights of the order 5000-30,000 Daltons. However, these low molecular weight gelatins exhibit inferior mechanical properties.

As mentioned above, gelatin is widely used for the micro-encapsulation of oils. These microcapsules are normally in the form of a granular powder or beadlets, and are formed by first emulsifying the oil phase in gelatin solution and then spray-drying or spray-chilling (into a fluidised starch bed) the emulsion, such as described eg in U.S. Pat. No. 5,120,761. The ability of the gelatin to stabilise the emulsion is an important feature. The gelatin may be extended by the inclusion of sugars or dextrins, to lower the cost of the product. The gelatin is responsible for the barrier function of the microcapsule walls, which prevent air oxidation, and it also provides mechanical strength such that the microcapsules may be compressed to form tablets without breakage. Both gelling gelatins and partially-hydrolysed gelatins may be used, but there is a minimum molecular weight below which the emulsification properties and the microcapsule wall strength become unsatisfactory; U.S. Pat. No. 5, 120,761 indicates a lower limit of 15,000 Daltons.

Despite the outstanding properties exhibited by gelatin, alternatives to gelatin are currently being sought, particularly in the pharmaceutical industry. This is partly due to religious and vegetarian pressures, which have created a desire to move to non-animal based products. Unsubstantiated concerns over gelatin presenting a potential risk from BSE (bovine spongiform encephalopathy) have also fuelled interest in alternatives.

To some extent, the desire to move from mammalian gelatin can be satisfied by using gelatin derived from fish collagen, but this does not satisfy vegetarians and, in any case, fish gelatin is commercially available in limited amounts, because of limited raw material supplies worldwide. Ideally, alternatives to gelatin should be of natural origin and non-animal based. Essentially, this means vegetable-derived materials.

To meet this requirement, hard capsules have been successfully produced using hydroxypropyl methylcellulose (HPMC) as a replacement for gelatin, as described in U.S. Pat. Nos. 5,264,223 and 5,431,917. The lack of gelling ability of HPMC has been compensated for by the inclusion of a gelling agent, carrageenan, together with a gelling aid (potassium chloride). Whilst it is claimed that such hard capsules show many of the desirable characteristics of conventional gelatin hard capsules and, indeed, some benefits, it is understood that they lack the desirable clear, glossy appearance. Moreover, HPMC is a chemically—modified cellulose, and therefore cannot be considered to be a natural product, but rather a food additive.

An alternative to the conventional rotary-die process for producing soft capsules has recently been described in PCT patent specification no.WO 97/3553. This avoids the use of gelatin (and also avoids the use of solutions) by using—directly—pre-formed films of polymer materials and applying solvent to the film to assist heat-sealing of the capsule walls. The preferred material is stated to be polyvinyl alcohol (PVA), preferably plasticised with glycerine. However, this synthetic polymer material is unsuitable for production of capsules for ingestion and is restricted to the production of soft capsules for technical applications. Other polymer film materials claimed to be usable in this process are alginate, HPMC, polyethylene oxide, polycaprolactone and pre-gelatinised starch. Of these, only alginate and pre-gelatinised starch can be described as natural, vegetable-derived materials. No information is provided on the appearance of capsules made using such materials or their suitability for the purpose, such as mechanical properties.

Recently, soft capsules based on potato starch plasticised with traditional polyalcohols have been described in the sales literature. dated Jul. 27, 2000, of Swiss Caps AG. Extruded material is used to feed conventional rotary-die machines. The soft capsules are claimed to have a smooth and shiny surface, but lack clarity and have poor mechanical properties (ie become brittle) at temperatures below 5° C.

PCT patent specification no.WO 98/26766 discloses the use of prolamines of vegetable origin to form films for encapsulation, as replacements for gelatin. It is not stated whether the films formed are clear. Prolamines are a class of proteins which are found only in cereals and are insoluble in water or neat alcohol, but are soluble in 50-90% alcohol and have relatively low molecular weights, of the order 10,000-40,000 Daltons. The preferred sources of prolamines are stated to be wheat and maize. According to PCT patent specification no. WO 97/10260, wheat gliadin (a prolamine) is a single-chain protein having an average molecular weight of approximately 30,000-40,000 Daltons. It is extremely sticky when hydrated and has little or no resistance to extension. The prolamine of maize (zein) has protein molecules with molecular weights covering the range 10,000-27,000 Daltons. The relatively low average molecular weights of the prolamines present limitations on the mechanical properties of the products produced from them.

Other vegetable proteins are commercially available in reasonably high purity, in the form of “isolates”, in which most of the carbohydrate present in the flour has been removed. Such isolates available include those derived from soya, wheat, pea and lupin. Also available are protein “concentrates”, which contain a lower proportion of protein. Such concentrates include those derived from soya, rice and maize. Technically, it would be possible to convert these concentrates into isolates by additional processing. Furthermore, there is a large range of protein-containing meals or flours, derived from various vegetable sources, which contain low levels of protein because carbohydrate has not been removed. Again, technically, these are capable of being converted into concentrates or isolates, using known procedures.

However, such vegetable protein isolates are unsuitable for use in capsule production, not least because they are not fully soluble in water. Even at alkaline pH, where such products may be claimed to have high solubility, ‘solubility’ in this context generally refers to resistance to separation when a dilute dispersion of the isolate is centrifuged. The dispersion in such products is not a clear solution. The solubility of isolates can often be increased by de-amidation and partial hydrolysis of the vegetable protein by acid or alkali treatment. However, such commercially-available products still do not form clear aqueous solutions.

By more extensive hydrolysis of vegetable proteins, using enzymes, acid or alkali, it is possible to achieve water-soluble protein hydrolysates, which produce clear films on drying. Such hydrolysates are widely used in the personal care industry as conditioning agents for skin and hair. However, they are unsuitable for capsule production since such films are weak and brittle, and lack mechanical strength. Typically, such hydrolysates have weight average molecular weights in the range 500-5000 Daltons.

There therefore remains a need for a natural, vegetable-derived, material capable of forming clear, mechanically strong products, as an alternative to or substitute for gelatin, particularly for edible and ingestible pharmaceutical applications.

The present invention overcomes many of the disadvantages, outlined above, of current gelatin alternatives for encapsulating applications by using high molecular weight, water-soluble proteins, derived from vegetable sources, which are capable of producing clear aqueous solutions and products of suitable mechanical strength, and are therefore suitable for use in known methods for the preparation of hard and soft capsules, and microcapsules.

Accordingly, the present invention provides a protein of vegetable origin suitable for use in capsule and microcapsule manufacture, which protein

(a) has a molecular weight of at least 40 KD

(b) is water-soluble, whereby a clear aqueous solution can be formed that can produce a clear film on drying.

In another aspect, the present invention provides the use of a protein of vegetable origin suitable in capsule or microcapsule manufacture, which protein

(a) has a molecular weight of at least 40 kD; and

(b) is water soluble, whereby a clear aqueous solution can be formed that can produce a clear film on drying.

In still another aspect, the present invention provides the use of a protein of vegetable origin suitable in capsule or microcapsule manufacture, which protein

(a) has a molecular weight of at least 40 kD; and

(b) is water soluble, whereby a clear aqueous solution can be formed that can produce a clear film on drying.

The water-soluble proteins of use in this invention preferably have weight average molecular weights of at least 50,000 Daltons, more preferably for soft and hard capsules, above 100,000 Daltons and, especially, above 200,000 Daltons. A particularly suitable molecular weight range is therefore 250,000 Daltons to 500,000 Daltons. These average molecular weight values are based on a size-exclusion HPLC procedure. Since there is no universally-accepted test method for determining average molecular weights of proteins and different methods can give different values, it is necessary to specify certain details of the test conditions used, in relation to the stated minimum average molecular weights of the proteins of this invention. These are:

Size exclusion column: TSK G4000 SWXL (30 cm×7.8 mm internal diameter)

Pump: Hewlett Packard HP1100 series isocratic pump (G1310A)

Injector: Hewlett Packard HP1100 series autosampler (G1313A)

Thermostat: Hewlett Packard HP1100 series thermostatted column compartment (G1316A)

Detector: Hewlett Packard HP1100 series variable wavelength detector (G1314A)

Control: Hewlett Packard HP1100 series Chemstation software (G2170AA)

Integration: Polymer Laboratories Caliber GPC software

Eluent: 0.05M KH₂PO₄, 0.05M K₂HPO₄,3H₂O and 0.1M NaCl adjusted to pH 7.0

Temperature: 25° C.

Detector wavelength: 220 nm

Calibration molecular weight standards: Sodium polystyrene sulphonate with molecular weights covering the approximate range 5000 Daltons to 1 million Daltons (Polymer Laboratories).

Preferably, the molecular weight of the protein is such as to enable the formation of a stable emulsion that can be processed according to the required end-use.

The specific, high molecular weight soluble proteins of this invention can be produced by a variety of processing routes known to those skilled in the art. Such processes may include controlled hydrolysis of the native vegetable protein using acid, alkali or enzymes, or a combination of these, followed by techniques to remove lower molecular components and selective recovery of components having weight average molecular weights in excess of 40,000 Daltons. Such separation processes may include selective precipitation, based on the relationship between molecular weight and solubility, dialysis or ultrafiltration.

Alternatively, the high molecular weight soluble proteins may be produced by a combination of hydrolysis and cross-linking reactions. The latter may include the controlled use of the enzyme transglutaminase, which is capable of forming cross-links between glutamine and lysine residues present in the protein chains, thereby increasing the average molecular weight. Other cross-linking routes that may be used include disulphide exchange reactions in which cystine residues present in the protein chains are broken and reformed to create larger protein chains. Examples of disulphide bond breakers are sodium thioglycollate and sodium bisulphite. Examples of disulphide bond re-formers are hydrogen peroxide and sodium bromate.

Other approaches to cross-linking to increase average molecular weight include heat treatment of the dry protein: for example, by heating at 80° C. in 90% RH environment for several hours. In such cases, separation of low molecular weight components and reaction products will normally still be necessary.

To achieve products that form clear solutions and dry to form clear films, clarification techniques may be used. Such techniques may include filtration, ultrafiltration and centrifugation. The use of filtration aids such as diatomaceous earth or chemical clarification, where haze-forming components are coagulated by addition of clarifying agent, may be necessary.

The preferred protein staring materials are ‘isolates’, since they contain the highest protein content. However, protein ‘concentrates’ and protein meals can also be used, although removal of carbohydrate may be necessary as a pre-treatment stage.

Examples of suitable vegetable-derived protein raw materials include, but are not limited to, wheat, soya, maize, rice, lupin, potato, jojoba, rape, pea, apricot kernel and evening primrose.

Examples of high molecular weight, soluble vegetable proteins currently available are Tritisol™ and Tritisol XM™, sold by Croda Oleochemicals of Cowick Hall, Snaith, Goole, E Yorkshire DN14 9AA, UK. These have an average molecular weight of approximately 250,000 Daltons and 500 KD, respectively, and are currently used as conditioning additives in both skin and hair care applications.

Surprisingly, we have found that these Tritisol™ proteins can be used to replace gelatin as an encapsulant in the production of soft capsules and microcapsules. Moreover, because Tritisol™ are derived from vegetable sources, they are edible, provided that chemical preservatives are not used or are first removed.

Unlike the ‘film-forming’ behaviour required to condition skin or hair, which can be achieved even with liquid films, a gelatin-replacement for capsules must be capable of producing a discrete container which combines properties of tensile strength and resilience with the ability to be heat-sealed and, preferably, form clear capsule walls. In the case of microcapsules, a gelatin-replacement must be capable of producing micro-containers with sufficient strength to be compressible into tablets, without significant leakage of the oil content.

Therefore, it is not possible to use all types of film-forming agent in the formation of capsules. Chambers Science and Technology Dictionary (1998) describes films as any thin layer of substance (eg a thin layer of material deposited, formed or adsorbed on another, down to mono-molecular dimensions). So, for example, in the personal care industry, various types of film-formers are used, which would not be suitable to replace gelatin in capsule manufacture, such as waxes (eg paraffin wax and microcrystalline wax), synthetic emollients (eg long-chain esters and fatty alcohols), clays, silicas, gums, resins, modified starches, modified cellulose and synthetic polymers.

However, for capsule production, the protein must be capable of forming a container having mechanical integrity, flexibility and resistance to compression. These properties are required to fulfill the requirements for established capsule manufacturing processes and also to exhibit the required resilience and robustness of the finished capsules. Clarity is important, largely for aesthetic reasons, and water-solubility is also an important feature. With such high molecular weight, water-soluble proteins, it is recognised that the maximum possible solution concentration will be limited by the viscosity of the solution, similar to the case for gelatin where it is not possible to achieve solution concentrations much higher than 50% due to viscosity restrictions.

The properties of the described high molecular weight soluble vegetable proteins may be modified and enhanced to suit any particular application by addition of other materials, as appropriate.

Unlike gelatin, these high molecular weight, soluble, vegetable derived proteins do not form heat-reversible elastic gels on cooling of solutions. Instead, they may exhibit gelling ability on heating above a critical temperature (eg 55° C.), but these gels are generally irreversible and nonelastic. For applications where the gelling properties are traditionally important, such as hard capsule manufacture, it may be necessary either to add vegetable-derived gelling agents, such as carrageenan or alginate or, more preferably, to use alternative technology, such as the use of pre-formed films of the protein or injection moulding techniques.

To improve the flexibility and increase the suppleness of the products formed from these proteins, the addition of plasticisers may be desirable. Examples of suitable plasticisers include glycerine, sorbitol, xylitol and propylene glycol. For example, during extrusion processes, the plasticiser may be present in the dry protein fed to the extruder (eg by spray drying protein plus plasticiser) or added to the protein in the extruder. It is envisaged that, for the manufacture of soft capsules, plasticised films, either pre-formed or extruded as part of the encapsulation process, are fed to conventional rotary die capsule machines to produce heat-sealable capsule walls, without the need to add water.

For encapsulation, eg micro-encapsulation, of food, cosmetic or pharmaceutical products, standard techniques known in the art, such as spray-drying an emulsion of the vegetable protein-derived gelatin substitute according to this invention onto a standard composition of the food, cosmetic or pharmaceutical. Alternatively, specially-designed processes may be used for micro-encapsulation.

Accordingly, the present invention further provides a food, cosmetic or pharmaceutical product comprising a food, cosmetic or pharmaceutical ingredient encapsulated in a vegetable protein-derived gelatin substitute, such as a protein identified or identifiable by the trademarks Tritisol or Tritisol XM.

In order that the invention may be more fully understood, the following examples are given by way of illustration only.

### EXAMPLE 1

High mwt Vegetable Protein Films

Films were cast from approximately 10% clear protein solutions (see Table 1), using the equivalent of 5 g dry solids, in Petri dishes. The films were dried in air under ambient conditions before removing from the dishes and subjectively assessing their characteristics.

TABLE 1

| | Weight average molecular | |
|---|---|---|
| Protein Source | weight (Daltons) | Film characteristics |
| Wheat | 395,550 | Clear, yellow, brittle, shiny |
| Wheat | 217,650 | Clear, yellow, brittle, Shiny |
| Wheat | 95,000 | Clear, yellow, brittle, shiny |
| Lupin | 169,740 | Clear, yellow, brittle, shiny |
| Lupin | 113,500 | Clear, yellow, brittle, shiny |
| Potato | 55,100 | Clear, amber, brittle, shiny |
| Rice | 141,500 | Clear, yellow, brittle, shiny |
| Maize | 87,600 | Clear, amber, brittle shiny |
| Jojoba | 67,480 | Clear, dark-brown, brittle |
| | | shiny |

All solutions formed were clear, superficially, the majority of films had the appearance of a gelatin film, apart from the colour, which varied from yellow through amber to dark brown. When flexed or extended, these films lacked the characteristic flexibility and extensibility of gelatin films, indicating the desirability of plasticising for certain applications. For a given protein source, the brittleness of the film was seen to show some decrease with increasing molecular weight.

All films were found to disintegrate then dissolve when immersed in water at 25° C.

### EXAMPLE 2

High mwt Wheat Protein Derived Films with Plasticiser

Films were cast, as in Example 1, using soluble wheat protein with a weight average molecular weight of 395,550 Daltons but with the addition of varying amounts of glycerol. On total solids, glycerol additions represented, respectively, 5, 10, 12.5, 15, 17.5 and 20%. The films were dried and equilibrated at 40%RH and approximately 20° C. and assessed subjectively for mechanical properties.

Increasing glycerol content progressively converted the film from being hard and brittle to flexible and extensible through to soft and weak. The film properties most closely matching those of a gelatin soft capsule wall film were achieved from a glycerine content of about 15-20%.

### EXAMPLE 3

Extruded High mwt Wheat Protein Plasticised Films

A solution of soluble wheat protein with a weight average molecular weight of 95,000 Daltons, was mixed with 20% by weight of glycerine (on protein solids) and spray dried to produce an agglomerated powder. The powder was fed via a screw-feed hopper to a 16 mm diameter, twin-screw extruder of process length 26:1. The material was extruded at a feed rate of 0.5 kg/hr and a heating temperature of 150° C. to give a transparent, flexible film, with a thickness of 0.18 mm.

The film was analysed and found to contain 16.4% glycerine and 8.6% moisture. It was found that the film could be heat-sealed. The film was shown to dissolve in water at 37° C.

### EXAMPLE 4

This followed the process of Example 3, except that soluble wheat protein powder with no added glycerine was used and mixed in the proportion 80:20 with glycerine in the extruder. Again, a clear flexible film was achieved, with a glycerine content of 21.3% and moisture content of 3.1%

### EXAMPLE 5

Effects of Relative Humidity (RH)

Sensitivity of the mechanical properties of the films to RH, due to tendency to pick-up or lose moisture, can be expected to be molecular weight dependent. Such changes are most likely to occur the lower the average molecular weight.

A soluble wheat protein, with weight average molecular weight of 51,000 Daltons was used to cast films in Petri dishes, as described in Example 2, except that glycerine contents of 20, 25, 30 and 40% were used and each of the films conditioned, respectively, at either 20% RH or ambient.

There was no obvious difference in the appearance or mechanical properties of the films, which could be attributable to the difference in RH. However, at 30% glycerine the clear flexible film showed signs of becoming slightly sticky and at 40% glycerine, the film was too soft to be useful for soft capsule production. These data indicate an optimum content of the order 20-25% glycerine.

1. The use of a protein of vegetable origin suitable in capsule or microcapsule manufacture, which protein
(a) has a molecular weight of at least 40 kD; and
(b) is water soluble, whereby a clear aqueous solution can be formed that can produce a clear film on drying.

2. The use according to claim 1, wherein the protein has a weight average molecular weight of at least 50 kD.

3. The use according to claim 1, wherein the protein has a weight average molecular weight of at least 200 kD.

4. The use according to claim 1, wherein the protein has a weight average molecular weight in the range of from 250 to 500 kD.

5. The use according to any preceding claim, wherein the capsules are soft capsules suitable for replacing soft gelatin capsules.

6. The use according to any of claims 1 to 4, wherein the capsules are microcapsules suitable for use in the preparation of tablets.

7. The use according to any of claims 1 to 4, wherein the capsules are hard capsules suitable for replacing hard gelatin capsules.

8. A capsule or microcapsule suitable for pharmaceutical or food use, comprising a protein of vegetable origin suitable in capsule or microcapsule manufacture, which protein
(a) has a molecular weight of at least 40 kD; and
(b) is water soluble, whereby a clear aqueous solution can be formed that can produce a clear film on drying.

9. A capsule or microcapsule according to claim 8, wherein the protein has a weight average molecular weight of at least 200 kD.

10. A capsule or microcapsule according to claim 8 or claim 9, further comprising a gelling agent, such as carrageenan or an alginate.

11. A capsule or microcapsule according to any of claims 8 to 10, further comprising a plasticiser, such as a glycerine derivative, sorbitol, xylitol or propylene glycol.

12. A capsule of microcapsule according to claim 11, comprising a wall film having a glycerine derivative content in the range of from 15 to 25% w/w, based on the total weight of the solids comprising the wall film.

13. A protein of vegetable origin suitable for use in capsule and microcapsule manufacture, which protein
(a) has a molecular weight of at least 40 kD; and
(b) is water soluble, whereby a clear aqueous solution can be formed that can produce a clear film on drying other than those identified or identifiable by the trademarks Tritisol and Tritisol XM.

14. A protein according to claim 13, having a weight average molecular weight of at least 200 kD.

15. A protein according to claim 13 or claim 14, wherein the vegetable is selected from wheat, soya, maize, rice, lupin, potato, jojoba, rape, pea, apricot kernel or evening primrose.

16. A food, cosmetic or pharmaceutical product comprising a food, cosmetic or pharmaceutical ingredient encapsulated in a protein according to any of claims 13 to 15 or a protein identified or identifiable by the trademarks Tritisol or Tritisol XM.
